(19) United States
(12) Patent Application Publication
(10) Pub. No.: US 2015/0150784 A1
(43) Pub. Date: Jun. 4, 2015

(54) DERMATOLOGICAL COMPOSITION CONTAINING CULTURED THEOBROMA CACAO CELLS OR ITS EXTRACTS AND RELATED METHODS

(71) Applicants: Sung-Yong H. Yoon, Lake Oswego, OR (US); Marc Philouze, West Linn, OR (US); Colby G. Caldwell, Oregon City, OR (US); Raymond E. B. Ketchum, Tigard, OR (US)

(72) Inventors: Sung-Yong H. Yoon, Lake Oswego, OR (US); Marc Philouze, West Linn, OR (US); Colby G. Caldwell, Oregon City, OR (US); Raymond E. B. Ketchum, Tigard, OR (US)

(21) Appl. No.: 14/406,731

(22) PCT Filed: Jun. 26, 2013
(86) PCT No.: PCT/US2013/048002

§ 371 Date: Dec. 9, 2014

Related U.S. Application Data
(60) Provisional application No. 61/664,741, filed on Jun. 26, 2012.

(51) Int. Cl. A61K 8/97; A61K 36/185; A61K 45/06; A61K 35/66

(52) U.S. Cl. CPC A61K 8/97; A61K 35/66; A61K 36/185; A61K 45/06; A61K 2800/591

(57) ABSTRACT

A dermatological composition for inhibiting or treating inflammation and/or aging of skin includes a dermatologically compatible component and cultured cells derived from the Theobroma cacao plant or an extract thereof. The cultured cells including anti-oxidant and/or anti-inflammatory compounds that reduce inflammation and/or aging when delivered to the skin.

### CROSS-REFERENCE TO RELATED APPLICATIONS

This application claims the benefit of U.S. Provisional Patent Application No. 61/664,741, titled, “Dermatological Composition Containing Theobroma Cacao Suspension Cells Or Its Extracts,” filed Jun. 26, 2012, which is hereby incorporated herein by reference.

### BACKGROUND

1. Field of the Invention

The present invention relates to the dermatological compositions that include cultured cells of Theobroma cacao. The cultured cells include increased concentrations of anti-inflammatory and anti-oxidant compounds as compared to native plant tissues.

2. Related Technology

Inflammation is a local response which occurs at injury sites to initiate the removal of pathogens or damaged tissues and plays an important role in various diseases, such as cardiovascular diseases, atherosclerosis and asthma. During an inflammatory response, mediators, such as pro-inflammatory cytokines, including interleukin IL-1, tumor necrosis factor (TNF), interferon (INF)-γ, IL-6, IL-12, IL-18 and the granulocyte-macrophage colony-stimulating factor, are released. This response is antagonised by anti-inflammatory cytokines, such as IL-4, IL-10, IL-13, IFN-a, and the transforming growth factor (Mueller, M.; Hobiger, S.; Jungbauer, A., Anti-inflammatory activity of extracts from fruits, herbs and spices. Food Chem 2010, 122, 987-996).

The nuclear factor-κB (NF-κB), transcription factor, also plays an important role in the inflammatory response by regulating the expression of various genes encoding pro-inflammatory cytokines, adhesion molecules, chemokines, growth factors, and inducible enzymes such as cyclooxygenase-2 (COX-2) (Hanada, T., & Yoshimura, A. Regulation of cytokine signaling and inflammation. Cytokine and Growth Factor Reviews 2002, 13(4-5), 413-42; Makarov, S. S. NF-kb as a therapeutic target in chronic inflammation: Recent advances. Molecular Medicine Today 2000, 6(11), 441-448). Inducible nitric oxide synthase (iNOS) and COX-2 both stimulate the production of large amounts of pro-inflammatory mediators.

In chronic inflammation, the negative regulatory mechanism appears to be dysfunctional. Although inflammation is primarily a protective response (against micro-organisms, toxins or allergens, for example), inflammation that is chronic and uncontrolled becomes detrimental to tissues (Gil, A. Polyunsaturated fatty acids and inflammatory diseases. Biomedicine and Pharmacotherapy 2002, 56(8), 388-396).

### BRIEF DESCRIPTION OF THE DRAWINGS

FIG. 1 shows the cell viability of cells after treatment by different concentrations of extract from cultured cells (i.e., Cocoa Cell Extract) compared to an extract from cocoa beans (i.e, Standard Cocoa Extract); and

FIG. 2 shows the effect of cocoa cell extract and standard cocoa extract on keratinocytes IL-8 release after stimulation by a mix of cytokines (IL-17+OSM+TNF-α).

### DETAILED DESCRIPTION

### I. Introduction

The present invention relates to the production and use of Theobroma cacao suspension cells and/or their extracts in dermal composition to inhibit dermal inflammation, aging, and/or oxidation. The cultured cells derived from Theobroma cacao are enriched in anti-oxidation, anti-inflammation and anti-aging compounds and impart these characteristics to the dermatology compositions.

The isolated cocoa cell line may be derived from essentially any part of the cocoa plant. For example, the isolated cocoa cell line may be derived from at least one of floral tissue or a non-floral vegetative tissue. Examples of floral tissue include, but are not limited to, petals, sepals, staminodes, and combinations thereof. Examples of non-floral vegetative tissue include, but are not limited to, nodes, internodes, young leaves, mature leaves, stems, roots, and combinations thereof.

The cells are derived from the Theobroma cacao plant and grown in cell culture. The cultured cell line is selected for its ability to produce therapeutically relevant concentrations of anti-aging, anti-inflammatory, and/or anti-oxidizing compounds in cell culture. For example, the isolated cultured cell line may include flavonoids such as (−)-epicatechin, (+)-catechin, and procyanidins, which are oligomers derived from these monomers, polyphenols such as quercetin, isoquercetin (quercetin 3-O-glucoside), quercetin 3-O-arabinose, naringenin and the like.

Effective amounts of anti-inflammatory and/or anti-oxidation compounds include cells having at least 5%, 10%, 20%, or 30% polyphenols and included in the dermatological compositions in concentrations of at least 0.01%, 0.1%, 0.5% 1%, 5%, or 10% by weight or in the case of the extract, the same or half the foregoing amounts.

In some embodiments of the invention, the dermatological compositions may be administered to a person identified as being in need of treatment or prevention of inflammation or aging of the skin. The method further includes administering to the skin a therapeutically effective amount of the dermatological composition. Studies show that the cultured cells or extracts can inhibit the production of proteins involved in an inflammatory response from exposure to nickel, thereby providing a therapeutic effect in subjects suffering from inflammation or aging of the skin.

The cultured cells can be grown rapidly in relatively large quantities and to high densities. In one embodiment, the isolated cocoa cell line yields greater than 100 mg/L packed cell volume (“PCV”), greater than 200 mg/L PCV of procyanidins, greater than 300 mg/L PCV, greater than 400 mg/L PCV, or greater than 500 mg/L PCV. In one embodiment, the isolated cocoa cell line yields greater than 100 mg, greater than 200 mg, or at least 250 mg of procyanidins per liter of cell culture.

The use of cell cultures for producing anti-inflammatory and anti-oxidant compounds has substantial advantages over cultivating the traditional plant and harvesting compounds from the bean. First, the cultured cells can be grown under conditions that stimulate the production of the desired compounds even if those conditions are not suitable for the cultivated native cocoa plant. Thus, cultivated cells provide an opportunity to naturally produce concentrations of compounds that are not possible in the cultivated plant. Secondly, the processing or extraction of the cultivated cells does not require the burdensome task of processing cocoa beans, which often results in producing undesired pigments or damaging or contaminating the desired compounds. Also, cell cultures are not subject to environmental conditions that can disrupt crop production or restricted to particular growing environments or growing seasons.

### II. Dermatological Compositions

The cultured cells are included in a dermatological composition to impart he beneficial effects of anti-inflammation and anti-oxidation. The dermatological composition may include any formulation of dermatological compounds combined with cultured cells so long as the combination is suitable for application to the skin of a person or animal in need of reduced inflammation or aging.

The dermatological composition may include cultured Theobroma cacao cells in a concentration of greater than and/or equal to 0.01%, 0.1%, 0.5% or 1% by weight or less than or equal to 30%, 20%, 10%, 5%, or 1% by weight or within a range of the foregoing upper and lower concentrations.

Alternatively, the dermatological composition may include an extract of the cultured Theobroma cacao cells. The concentration of the extract may be at least 0.0001, 0.001, 0.01, or 0.1 by weight and/or less than 10%, 5%, 1%, or 0.1% by weight or within a range of the foregoing upper and lower concentrations. Because the cultured cells produce unique secondary metabolite profiles, the extracts likewise have unique profiles as compared to extracts from the native cocoa bean. In particular, the extracts from the cultured cells have substantially increased percentages of anti-inflammatory and anti-oxidant compounds.

In addition to the cultured cells or extracts, the dermatological composition includes carriers for delivering the cultures cells or extracts to a person's skin. For example, the composition may be formulated into solution, suspension, emulsion, paste, gel, cream, lotion, powder, soap, surfactant-containing cleanser, oil, powder foundation, emulsion foundation, wax foundation, or a spray suitable for application to the skin, and other cosmetic products that have a similar function to the foregoing.

The composition can include solvents, carriers, and adjuvants that facilitate delivery and use of the composition to the skin. In one embodiment, the composition can include fatty alcohols, glycols, cholesterol, pH modifying agents, and the like.

More specifically, the cultured cells may be formulated into emollient lotion, nourishing lotion, nourishing cream, massage cream, essence, eye cream, cleansing cream, cleansing foam, cleansing water, pack, spray or powder.

When the composition is formulated as a paste, cream or gel, the composition may include animal oil, plant oil, wax, paraffin, starch, tragacanth, cellulose derivatives, polyethylene glycol, silicone, bentonite, silica, talc, zinc oxide, etc.

When the composition is formulated as a powder or spray, the composition may include lactose, talc, silica, aluminum hydroxide, calcium silicate or polyamide powder. In particular, a spray composition may include a propellant such as chlorofluorohydrocarbon, propane/butane or dimethyl ether.

For a solution or emulsion, the composition may include a solvent, solubilizer, or emulsifier. Examples include, water, ethanol, isopropanol, ethyl carbonate, ethyl acetate, benzyl alcohol, benzyl benzoate, propylene glycol, 1,3-butylglycol oil, glycerol aliphatic ester, polyethylene glycol or fatty acid ester of sorbitan may be used.

For a suspension, the composition may include a liquid diluent such as water, ethanol or propylene glycol, a suspending agent such as ethoxylated isostearyl alcohol, polyoxyethylene sorbitol ester and polyoxyethylene sorbitan ester, microcrystalline cellulose, aluminum metahydroxide, bentonite, agar, tragacanth, and the like.

For a surfactant-containing cleanser, the composition may include aliphatic alcohol sulfate, aliphatic alcohol ether sulfate, sulfosuccinic monoester, isethionate, imidazolinium derivatives, methyl taurate, sarcosinate, fatty acid amide ether sulfate, alkyl amidobetaine, aliphatic alcohol, fatty acid glyceride, fatty acid diethanolamide, vegetable oil, lanolin derivatives, ethoxylated glycerol fatty acid ester, etc. may be used as a carrier. The dermatology composition may also include common adjuvants such as antioxidant, stabilizer, solubilizer, vitamin, pigment and/or fragrance.

### III. Producing Cultured Cells with Anti-Inflammatory and Anti-Oxidant Compounds

Methods provided herein can generate prolific cell cultures that produce relatively concentrated amounts of compounds with anti-inflamatory and/or antioxidative properties. Representative advantages of these methods include: reliable and continuous source of biomass due to control of climatic conditions; rapid and efficient isolation procedures that minimizes degradation of the anti-inflammatory and anti-oxidative compounds during the extraction process, as compared to materials compositions that can be isolated from cocoa beans using techniques known in the art.

In general, described herein is the establishment of callus cultures from various tissues of Theobroma plant. Established calli are used to raise suspension cultures using various types of cell culture media. When stable suspension cell cultures are established the cells are extracted and analyzed for anti-inflammatory and anti-oxidant content by HPLC-MS methods. From such analysis, suspension cultures capable of producing the desired anti-inflammatory and anti-oxidative compounds are selected for further optimization of productivity.

### Generation of Cocoa Culture

The process of generating cocoa cultures is generally outlined here, and detailed exemplary protocols are described in the Examples. Initiation of cell cultures producing the anti-inflammatory and anti-oxidatant compounds is achieved by establishing callus and suspension cultures from explants derived from any of various plant parts, for example, floral tissues such as petals, sepals, staminodes, etc., or non-floral vegetative tissues such as node, internode, young leaves, mature leaves, as described for somatic embryogenesis. The suspension cultures are maintained in fresh suspension culture medium by periodic transfer of a portion of the cultured cells to fresh medium. Transfer schedule and inoculum density is determined by cell growth performance and sugar consumption from the medium.

One embodiment provides a method for modifying the concentration of anti-inflammatory and anti-oxidant compounds, which involves initiating the cultures under conditions sufficient to produce the desired concentration of anti-inflammatory or anti-oxidant compounds and establishing a production medium sufficient to establish productive cell cultures, followed by scaling up of the productive cell cultures for an appropriate amount of time to produce the desired concentration of the compounds. Accordingly, altering the conditions required to initiate a culture or establish productive cultures results in modified content (amount) of desired anti-inflammatory and/or anti-oxidant compounds in such cultures. Physical aspects (e.g., light irradiance) and/or chemical aspects (e.g., media composition or chemical elicitors) of the plant cell culture microenvironment may be varied to achieve the desired modified content.

For instance, carbohydrate (e.g. sucrose or glucose) concentration in a suspension medium may be increased initially or during elicitation in order to increase the amount of procyanidins in the culture. Furthermore, nitrogen sources (e.g., ammonium nitrate) may be manipulated for production of secondary metabolites in plant cell cultures (see Neera et al., Phytochemistry. 31(12): 4143-4149, 1992). In addition, infusion of certain amino acids (such as glutamine, glycine, and serine) also may significantly affect the production of secondary metabolites. As a result, the concentrations of these amino acids in Theobroma suspension medium may be increased in order to enhance the production of anti-inflammatory and/or anti-oxidant compounds. In addition, hormones may be removed from culture medium to enhance production of the desired compounds in suspension cultures.

Lighting conditions also can be varied in order to achieve modified content of anti-inflammatory and/or anti-oxidant compound in the cell cultures. For example, the lighting can be changed by increasing irradiance or length of exposure to the light. The wavelength of the light irradiance can also be changed.

Other modifications known in the art for manipulation of plant cell culture microenvironments are also contemplated as being within the scope of the present description.

Additionally, the cultured cells can be dosed with glucose at some point prior to harvesting. The glucose may be substituted with other sugar such as sucrose, fructose, or the like. The glucose can be introduced into a cell culture: at the time of seeding; 1-7 days after seeding; 7-14 days after seeding; 14-21 days after seeding; or combinations thereof. In another example, the cultures can be grown in a hormone-free medium. The hormone-free medium can be introduced to the cells in culture: at the time of seeding; 1-7 days after seeding; 7-14 days after seeding; 14-21 days after seeding; or combinations thereof.

In some embodiments the cultured cell line may be selected to have little or no pigment. For example, the cultured cells may be substantially free of tannins and/or anthocyanins that are typical of cocoa bean products. In one embodiment, the cultured cells and/or the extracts thereof have less than 2%, 1%, 0.5% tannins and/or less than 1%, 0.5%, 0.1%, or 0.01% anthocyanins.

Harvesting of Cocoa Cells from Culture

A quantity of cultured cells of Theobroma producing anti-inflammatory or anti-oxidant compounds are grown as described herein, and cells are harvested to isolate the cells from the culture media. Harvesting of suspension cells can be performed in a number of ways.

Once a cell culture has reached stationary phase and the desired productivity of compounds is reached, the culture is allowed to settle as a compact mass in the container and the medium can be decanted, leaving behind the mostly solid cell biomass. The cell biomass is then washed to remove remaining medium and similarly decanted. Alternatively, the cell suspension can be centrifuged and the supernatant (medium) discarded followed by washing of the cell mass and centrifugation again to discard the liquid. A third option is to filter the cell culture suspension to remove the medium. Any of these methods can be employed with cell culture volumes ranging from a few milliliters to production scale volumes of greater than 100 L, 1000 L, 5000 L, or even greater than 10,000 L.

### Extraction Procedures

The harvested cell mass is crushed, milled, or ground to homogenize the cell mass and break up the cells to enhance the surface area with extraction solvent with the sample, and to ensure that the extracted portion is representative for the entire sample.

Extraction of total polyphenols from cell cultures of Theobroma is similar to procedures used for extracting total polyphenols from cocoa beans, except for a few major differences. In case of cocoa beans, the initial step after grinding the beans is to defat the ground flakes (nibs). This process results in a loss of polyphenols. Since cell cultures do not have as much fat as the beans, this step is not required. Removing this step reduces the loss of polyphenols during the extraction process from a cell culture.

Furthermore, defatting requires solvents such as hexane, and traces of the solvent are found in the final extract. This causes an unpleasant odor in extracts produced from cocoa beans, and the solvents may be toxic or undesirable for certain uses, such as a food ingredient. Since the methods described herein for extraction from cell culture biomass eliminate the use of solvents (such as hexane), there is no solvent contamination or unpleasant solvent odor in the resultant extract.

In one example, anti-inflammatory and/or anti-oxidant compounds may be extracted from ground, homogenized cells with 70-80% aqueous methanol or 70% aqueous acetone, or combinations thereof. Water and ethanol have also been used, though oligomeric procyanidins are extracted only partially using these solvents, and high molecular weight polymers are not extracted at all (Grayer, In J. B. Harborne, Plant Phenolics (Vol. 1), pp 283-323, 1989. San Diego, Academic Press, Inc.; Lee & Widmer, In L. M. L. Nollet, Handbook of Food Analysis (Vol. 1), pp 821-894, 1996, Basel, New York, Hong Kong, Marcel Dekker, Inc.).

In one embodiment, the extraction solvent can include ethanol. The ethanol can be an aqueous composition that includes from about 25% to about 100% ethanol by weight or volume, more preferably more than about 50%, more preferably more than 75%, and even more preferably more than 90%. Specific examples include 50% ethanol, 60% ethanol, 70% ethanol, and 80% ethanol, where FIG. 12 shows 60% ethanol being superior. In this embodiment, the ethanol extraction solvent is devoid of other alcohols or ketones.

In one embodiment, the extraction solvent can include other alcohols or ketones along with water and/or ethanol, such as aqueous organic solvents. Examples include isopropyl alcohol, ethanol, methanol, acetone, ethylacetate or a combination thereof. The aqueous organic solvents can include water from about 25% to about 99%, more preferably from about 30% to about 90%, or even more preferably more than 50% water.

In one embodiment, the extraction solvent is combined with the processed or unprocessed cells at various amounts depending on the amount of cell mass. The amount of extraction solvent can be 50% or more of the mass or volume of the cell mass, more preferably more than or about 75%, even more preferably more than or about 100%, and even more preferably more than 200% of the mass or volume of cell mass. Of course, larger volumes of extraction solvent can be used.

In one embodiment, the extraction solvent can include an acid, such as an antioxidant acid. Examples of acids include acetic acid, citric acid, or ascorbic acid. The acid can be present from about 0.05% to 10% by volume of the extraction composition or by volume of the extraction solvent, more preferably from about 0.75% to about 5%, or most preferably from about 0.1% to about 1% or to about 2% by volume of the extraction composition or by volume of the extraction solvent.

Additional details regarding culturing Theobroma cocoa can be found in Applicant's co-pending U.S. application Ser. No. 13/251,960 filed Oct. 3, 2011, which is hereby incorporated herein by reference in its entirety. The embodiments of the foregoing application may be used in combination with the various features of the inventions described herein.

### IV. Examples

### Example 1

### Preparation of Theobroma cacao Suspension Cells

Vegetative tissue (nodes) derived suspension cultures were derived from calli and the best-performing cell line was selected and grown in Murashige and Skoog (MS) medium, supplemented with auxins (1 mg/L IAA and 2 mg/L IBA), cytokinins (0.005 mg/L TDZ), and L-Glutamine (250 mg/L) with Glucose (40 g/L) as the carbon source. Cell growth rate is significantly important for maximizing the volumetric productivity in cell culture process. Well-growing, fine cells that are not clumpy or forming aggregates were preferably selected at each subculture time to increase the volumetric productivity and eliminate aggregated cells, since selection of large or clumpy cell aggregates leads to poor culture performance. Therefore, the selected cells had mainly yellow-colored fine cell morphology and the fine suspension culture of homogeneous suspension cells resulted in stable growth and production.

### Example 2

### Extract Preparation of Suspension Cells of Theobroma Cacao Cultures

This example describes methods developed for extracting polyphenol compounds from suspension cells of T. cacao cultures. T. cacao fresh suspension cells without media or dried and ground T. cacao cells were resuspended in acetone:water (70:30, v/v), stirred for 1-3 hr at room temperature in dark. The suspension was centrifuged at 3000-5000 xg for 20 min to separate cell residue and the supernatant was collected. The same process was repeated with the cell residue for improving extraction efficiency. The solid phase was discarded and the supernatant was used for analysis or further purification processes. Then the supernatant was concentrated using a rotary vacuum concentrator and the concentrated sample was dried using a freeze-dryer. The dried sample was dissolved in DMSO, thereby obtaining a DMSO extract of T. cacao suspension cells for further tests.

### Example 3

### Preparation of Homogenized T. cacao Whole Cell Lysate

This example describes methods developed for preparing whole cell lysates of T. cacao suspension culture. T. cacao fresh cells without media or dried T. cacao cells were resuspended in 1˜50% ethanol (v/v), sonicated for 30˜90 min at room temperature in dark. The suspension was homogenized by a mechanical homogenizer and concentrated using a rotary vacuum concentrator to evaporate ethanol. Then DMSO was added to compensate the evaporated volume of ethanol, thereby obtaining a T. cacao whole cell lysate for further tests.

### Example 4

### Cytotoxicity test

In order to examine whether the T. cacao cell extract or T. cacao whole cell lysate is cytotoxic, the following test was carried out. Human diploid fibroblasts (HDF) cells were cultured in 10% FBS (fetal bovine serum)-containing DMEM (Dulbecco's Modified Eagle's Medium). The fibroblasts were seeded into a 96-well plate at a density of 1×10⁴ cells per well and cultured for 12 hours. Then, FBS-free DMEM medium was treated for 24 hours with each of 1˜500 mg/L of the T. cacao cell extract or T. cacao whole cell lysate. Untreated samples with the cell extract or the cell lysate were regarded as control. The viability of the fibroblasts was determined by culturing the cells for 2 hours in FBS-free DMEM medium containing 10% WST-1 solution and then measuring the absorbance at 450 nm. As a result, the T. cacao cell extract and the whole cell lysate did not reduce the cell viability in the concentration range used. Because a substance showing a cell viability of less than 80% is considered to be cytotoxic, it was confirmed that T. cacao suspension cell extract and the whole cell lysate were not cytotoxic in the concentration ranges used.

### Example 5

### Measurement of Anti-Inflammatory Activity of Extract from T. cacao Suspension Cell Extract and T. cacao Cell Lysate

Nickel is a silvery-white metal that can be found in nature. It is usually mixed with other metals to produce alloys. For example, nickel-iron, which is used to manufacture stainless steel, is the most common nickel alloy. Other nickel alloys are used to make coins, costume jewelry, bra or girdle fasteners, zippers, snaps, buttons, suspender clips, hair-pins, studs, eyeglass frames, pens, handles, utensils, paper clips, keys, and tools. A nickel allergy is a reaction that develops after initial and/or brief, or repeated and/or prolonged, exposure to nickel or nickel-containing items, depending on the individual's susceptibility. A nickel allergy can occur at any age, and typically manifests a few days after first contact as eczema (allergic contact dermatitis), which appears as an itchy, dry/crusty, and red/pigmented skin rash with watery blisters. The affected area is usually restricted to the site of contact, although it could also be found on other parts of the body. Once a nickel allergy has developed, it is usually a chronic condition, often being life-long. Nickel compounds are prime inducers of contact allergy reactions in humans.

Lipopolysaccharide (LPS), E. coli toxin and nickel activate nuclear factor-κB (NF-κB) in the nuclei of cells, such as macrophages, fibroblasts, dendritic cells and lymphocytes, to stimulate the secretion of inflammatory cytokines and cause allergic inflammation. This example describes T. cacao suspension cell extract and T. cacao cell lysate inhibit expression of proteins related to human skin inflammation and show effective anti-inflammatory activity.

5-1. Inhibition of Expression of Matrix Metalloproteinase-2 (MMP-2).

With the stimulus of LPS or nickel, MMP-2 is overexpressed and its product is increased by inflammatory signal transduction. HDF cells were treated with LPS and various volumes of T. cacao cell extract or T. cacao whole cell lysate and after 0˜24 hrs after the treatment, the HDF cells were collected every 6 hr to quantify MMP-2 expression by Western blotting. The quantified protein was mixed with bromophenol blue dye solution, and then subjected to 10% SDS-polyacrylamide gel electrophoresis (SDS-PAGE). After the electrophoresis, the protein was transferred to a polyvinylidene fluoride membrane (Millipore) and immersed in 0.5% skim milk-containing TBS (Tris buffered saline)-tween solution (10 mM Tris. HCl, 100 mM NaCl, 0.1% Tween 20, pH 7.5) to block nonspecific reactions. Then, the membrane was allowed to react with a 1:500 dilution of anti-mouse antibody for MMP-2 at room temperature for 3 hours, and then with anti-mouse IgG antibody as secondary antibody. After completion of the reaction, the membrane was washed 4 times with TBS (Tris buffered saline)-tween solution, and allowed to react with ECL (enhanced chemiluminescence) detection reagent for 1 minute, and then exposed to an X-ray film at room temperature. The results demonstrated that the MMP-2 was reduced in the samples treated by LPS with both T. cacao cell extract and lysate treatments.

5-2. Inhibition of Expression of Matrix Metalloproteinase-9 (MMP-9).

With the stimulus of LPS or nickel, MMP-9 is overexpressed and its product is increased by inflammatory signal transduction. HDF cells were treated with LPS and various volumes of T. cacao cell extract or T. cacao whole cell lysate and after 0˜24 hrs after the treatment, the HDF cells were collected every 6 hr to quantify MMP-9 expression by Western blotting. The quantified protein was mixed with bromophenol blue dye solution, and then subjected to 10% SDS-polyacrylamide gel electrophoresis (SDS-PAGE). After the electrophoresis, the protein was transferred to a polyvinylidene fluoride membrane (Millipore) and immersed in 0.5% skim milk-containing TBS (Tris buffered saline)-tween solution (10 mM Tris. HCl, 100 mM NaCl, 0.1% Tween 20, pH 7.5) to block nonspecific reactions. Then, the membrane was allowed to react with a 1:500 dilution of anti-mouse antibody for MMP-9 at room temperature for 3 hours, and then with anti-mouse IgG antibody as secondary antibody. After completion of the reaction, the membrane was washed 4 times with TBS (Tris buffered saline)-tween solution, and allowed to react with ECL (enhanced chemiluminescence) detection reagent for 1 minute, and then exposed to an X-ray film at room temperature. The results demonstrated that the MMP-9 was reduced in the samples treated by LPS with both T. cacao cell extract and lysate treatments.

5-3. Inhibition of IL-1β Expression.

With the stimulus of LPS or nickel, IL-1β is overexpressed and its product is increased by inflammatory signal transduction. HDF cells were treated with LPS and various volumes of T. cacao cell extract or T. cacao whole cell lysate and after 0˜24 hrs after the treatment, the HDF cells were collected every 6 hr to quantify IL-1β expression by Western blotting. The quantified protein was mixed with bromophenol blue dye solution, and then subjected to 10% SDS-polyacrylamide gel electrophoresis (SDS-PAGE). After the electrophoresis, the protein was transferred to a polyvinylidene fluoride membrane (Millipore) and immersed in 0.5% skim milk-containing TBS (Tris buffered saline)-tween solution (10 mM Tris. HCl, 100 mM NaCl, 0.1% Tween 20, pH 7.5) to block nonspecific reactions. Then, the membrane was allowed to react with a 1:500 dilution of anti-mouse antibody for IL-1β at room temperature for 3 hours, and then with anti-mouse IgG antibody as secondary antibody. After completion of the reaction, the membrane was washed 4 times with TBS (Tris buffered saline)-tween solution, and allowed to react with ECL (enhanced chemiluminescence) detection reagent for 1 minute, and then exposed to an X-ray film at room temperature. The results demonstrated that the IL-1β was reduced in the samples treated by LPS with both T. cacao cell extract and lysate treatments.

5-4. Inhibition of IL-8 Expression.

With the stimulus of LPS or nickel, IL-8 is overexpressed and its product is increased by inflammatory signal transduction. HDF cells were treated with LPS and various volumes of T. cacao cell extract or T. cacao whole cell lysate and after 0˜24 hrs after the treatment, the HDF cells were collected every 6 hr to quantify IL-8 expression by Western blotting. The quantified protein was mixed with bromophenol blue dye solution, and then subjected to 10% SDS-polyacrylamide gel electrophoresis (SDS-PAGE). After the electrophoresis, the protein was transferred to a polyvinylidene fluoride membrane (Millipore) and immersed in 0.5% skim milk-containing TBS (Tris buffered saline)-tween solution (10 mM Tris. HCl, 100 mM NaCl, 0.1% Tween 20, pH 7.5) to block nonspecific reactions. Then, the membrane was allowed to react with a 1:500 dilution of anti-mouse antibody for IL-8 at room temperature for 3 hours, and then with anti-mouse IgG antibody as secondary antibody. After completion of the reaction, the membrane was washed 4 times with TBS (Tris buffered saline)-tween solution, and allowed to react with ECL (enhanced chemiluminescence) detection reagent for 1 minute, and then exposed to an X-ray film at room temperature. The results demonstrated that the IL-8 was reduced in the samples treated by LPS with both T. cacao cell extract and lysate treatments.

5-5. Inhibition of IL-18 Expression.

With the stimulus of LPS or nickel, IL-18 is overexpressed and its product is increased by inflammatory signal transduction. HDF cells were treated with LPS and various volumes of T. cacao cell extract or T. cacao whole cell lysate and after 0˜24 hrs after the treatment, the HDF cells were collected every 6 hr to quantify IL-18 expression by Western blotting. The quantified protein was mixed with bromophenol blue dye solution, and then subjected to 10% SDS-polyacrylamide gel electrophoresis (SDS-PAGE). After the electrophoresis, the protein was transferred to a polyvinylidene fluoride membrane (Millipore) and immersed in 0.5% skim milk-containing TBS (Tris buffered saline)-tween solution (10 mM Tris. HCl, 100 mM NaCl, 0.1% Tween 20, pH 7.5) to block nonspecific reactions. Then, the membrane was allowed to react with a 1:500 dilution of anti-mouse antibody for IL-18 at room temperature for 3 hours, and then with anti-mouse IgG antibody as secondary antibody. After completion of the reaction, the membrane was washed 4 times with TBS (Tris buffered saline)-tween solution, and allowed to react with ECL (enhanced chemiluminescence) detection reagent for 1 minute, and then exposed to an X-ray film at room temperature. The results demonstrated that the IL-18 was reduced in the samples treated by LPS with both T. cacao cell extract and lysate treatments.

5-6. Inhibition of IL-22 Expression.

With the stimulus of LPS or nickel, IL-22 is overexpressed and its product is increased by inflammatory signal transduction. HDF cells were treated with LPS and various volumes of T. cacao cell extract or T. cacao whole cell lysate and after 0˜24 hrs after the treatment, the HDF cells were collected every 6 hr to quantify IL-22 expression by Western blotting. The quantified protein was mixed with bromophenol blue dye solution, and then subjected to 10% SDS-polyacrylamide gel electrophoresis (SDS-PAGE). After the electrophoresis, the protein was transferred to a polyvinylidene fluoride membrane (Millipore) and immersed in 0.5% skim milk-containing TBS (Tris buffered saline)-tween solution (10 mM Tris. HCl, 100 mM NaCl, 0.1% Tween 20, pH 7.5) to block nonspecific reactions. Then, the membrane was allowed to react with a 1:500 dilution of anti-mouse antibody for IL-22 at room temperature for 3 hours, and then with anti-mouse IgG antibody as secondary antibody. After completion of the reaction, the membrane was washed 4 times with TBS (Tris buffered saline)-tween solution, and allowed to react with ECL (enhanced chemiluminescence) detection reagent for 1 minute, and then exposed to an X-ray film at room temperature. The results demonstrated that the IL-22 was reduced in the samples treated by LPS with both T. cacao cell extract and lysate treatments.

5-7. Inhibition of IL-23 Expression.

With the stimulus of LPS or nickel, IL-23 is overexpressed and its product is increased by inflammatory signal transduction. HDF cells were treated with LPS and various volumes of T. cacao cell extract or T. cacao whole cell lysate and after 0˜24 hrs after the treatment, the HDF cells were collected every 6 hr to quantify IL-23 expression by Western blotting. The quantified protein was mixed with bromophenol blue dye solution, and then subjected to 10% SDS-polyacrylamide gel electrophoresis (SDS-PAGE). After the electrophoresis, the protein was transferred to a polyvinylidene fluoride membrane (Millipore) and immersed in 0.5% skim milk-containing TBS (Tris buffered saline)-tween solution (10 mM Tris. HCl, 100 mM NaCl, 0.1% Tween 20, pH 7.5) to block nonspecific reactions. Then, the membrane was allowed to react with a 1:500 dilution of anti-mouse antibody for IL-23 at room temperature for 3 hours, and then with anti-mouse IgG antibody as secondary antibody. After completion of the reaction, the membrane was washed 4 times with TBS (Tris buffered saline)-tween solution, and allowed to react with ECL (enhanced chemiluminescence) detection reagent for 1 minute, and then exposed to an X-ray film at room temperature. The results demonstrated that the IL-23 was reduced in the samples treated by LPS with both T. cacao cell extract and lysate treatments.

5-8. Inhibition of IL-31 Expression.

With the stimulus of LPS or nickel, IL-31 is overexpressed and its product is increased by inflammatory signal transduction. HDF cells were treated with LPS and various volumes of T. cacao cell extract or T. cacao whole cell lysate and after 0˜24 hrs after the treatment, the HDF cells were collected every 6 hr to quantify IL-23 expression by Western blotting. The quantified protein was mixed with bromophenol blue dye solution, and then subjected to 10% SDS-polyacrylamide gel electrophoresis (SDS-PAGE). After the electrophoresis, the protein was transferred to a polyvinylidene fluoride membrane (Millipore) and immersed in 0.5% skim milk-containing TBS (Tris buffered saline)-tween solution (10 mM Tris. HCl, 100 mM NaCl, 0.1% Tween 20, pH 7.5) to block nonspecific reactions. Then, the membrane was allowed to react with a 1:500 dilution of anti-mouse antibody for IL-31 at room temperature for 3 hours, and then with anti-mouse IgG antibody as secondary antibody. After completion of the reaction, the membrane was washed 4 times with TBS (Tris buffered saline)-tween solution, and allowed to react with ECL (enhanced chemiluminescence) detection reagent for 1 minute, and then exposed to an X-ray film at room temperature. The results demonstrated that the IL-31 was reduced in the samples treated by LPS with both T. cacao cell extract and lysate treatments.

### Example 6

### Measurement of Antioxidant Activity of T. cacao Suspension Cell Extract and T. cacao Whole Cell Lysate

Human diploid fibroblast (HDF) cells were used to examine whether reactive oxygen species (ROS) induced by H₂O₂ are inhibited by the T. cacao suspension cell extract and T. cacao whole cell lysate. The measurement of intracellular reactive oxygen species was carried out by FAC Scan analysis of 2′,7′-dichlorofluorescein diacetate (DCFDA) fluorescent dye. HDF cells incubated with DCFDA were washed twice in phosphate-buffered saline (PBS) and collected by treatment with trypsin-EDTA. Then, the cells were collected by centrifugation at 900 rpm for 5 minutes, and ROS per 10,000 cells were measured. The cells were dispensed into a 6-well plate and then treated with H2O2 alone or in combination with the T. cacao suspension cell extract and T. cacao whole cell lysate. Then the cells were washed 2-3 times with HBSS (Hank's balanced salt solution) and stabilized in HBSS for 30 min. DCFDA stained the cells under 37° C. for 1 hr, washed three times with HBSS and then observed with a fluorescence microscope. As a result, T. cacao suspension cell extract and T. cacao whole cell lysate inhibited the production of ROS.

### Example 7

### Inhibition of MMP-1 (Human Fibroblast Collagenase)

In order to measure the anti-aging activity of the T. cacao suspension cell extract and T. cacao whole cell lysate, fibroblasts were seeded into a 24-well plate at a density of 2×10⁴ cells per well and cultured for 12 hours to adhere to the culture plate. Then, the cells were starved in FBS-free DMEM medium for 12 hours. The cells were washed with DPBS buffer and irradiated with UV light at a dose of 100 mJ/cm² at 365 nm. Also, the DMEM medium was treated for 24 hours with each of 10˜50 ppm of the T. cacao suspension cell extract and T. cacao whole cell lysate. Also, a positive control group was treated with 1 μM RA (retinoic acid), and an additional group was treated with 10˜50 ppm of the T. cacao suspension cell extract or T. cacao whole cell lysate. Then, the medium was collected and centrifuged, and the amount of MMP-1 in the supernatant was quantified by an ELISA assay. By using WST-1 solution, the cell viability was measured and corrected. As a result, the T. cacao suspension cell extract and T. cacao whole cell lysate showed an excellent of inhibiting MMP-1 production and its relevant anti-aging activity.

### Example 8

### Inhibition of Melanogenesis

Murine melanoma cells (B-16 Fl) were added to 10% FBS-containing DMEM medium in a 6-well plate at a density of 1×10⁵ cells per well, and then cultured under conditions of 5% CO₂ and 37° C. until about 80% of the cells were attached to the well bottom. Then, the medium was replaced with a medium containing each of 1 and 10 ppm of the T. cacao suspension cell extract or T. cacao whole cell lysate, and the cells were cultured under 5% CO2 and 37° C. for a given time. Also, a positive control group was treated with 1 mM kojic acid, and a negative control group was not treated with the sample. The cells from which the medium has been removed was washed with PBS and collected by treatment with trypsin. The cell pellets were added to 100 μl 1 M NaOH containing 10% DMSO, thus obtaining intracellular melanin. The solution was measured for absorbance at 490 nm using a microplate reader, and the amount of melanin per protein was calculated. The protein was quantified by a Bradford assay. As a result, the T. cacao suspension cell extract and T. cacao whole cell lysate inhibited melanogenesis.

### Example 9

### Cosmetic Formulation

Example 9 provides a formulation of a cosmetic cream according to one embodiment of the invention.

| No. | Ingredient | Mass % |
|---|---|---|
| 1 | 1,3-Butylene Glycol | 5.0 |
| 2 | Ceramide | 1.5 |
| 3 | Cholesterol | 1.0 |
| 4 | Distilled Water | 72.4 |
| 5 | Glycol Monostearate | 4.0 |
| 6 | Lecithin | 1.0 |
| 7 | Potassium Hydroxide | 0.1 |
| 8 | Stearyl Alcohol | 3.5 |
| 9 | Stearic Acid | 2.5 |
| 10 | T. cacao dried and ground cells | 9.0 |

| | |
|---|---|
| Total | 100 |

### Example 10

### Dermatological Formulation

Example 10 provides an example dermatological formulation according to one embodiment of the invention.

| No. | Ingredient | Mass % |
|---|---|---|
| 1 | Beta sitosterol | 12.0 |
| 2 | Carboxyvinyl Polymer | 0.4 |
| 3 | Ceteareth-20 | 6.0 |
| 4 | Ceramide | 0.1 |
| 5 | Cholesterol | 0.3 |
| 6 | Concentrated Glycerin | 2.0 |
| 7 | DEA-cetyl Phosphate | 0.4 |
| 8 | Distilled Water | 60.15 |
| 9 | Fragrance | 0.15 |
| 10 | Macadamia Nut Oil | 10.0 |
| 11 | Polyglyceryl-2 Oleate | 0.2 |
| 12 | Preservative | 0.2 |
| 13 | T. cacao suspension cell extract | 8.0 |
| 14 | Xanthan Gum | 0.1 |

| | |
|---|---|
| Total | 100 |

### Example 11

### Functional Dermatological Ointment

Example 11 provides a formulation of a dermatological ointment according to one embodiment of the invention.

| No. | Ingredient | Mass % |
|---|---|---|
| 1 | Boric acid | 1.0 |
| 2 | Zinc oxide | 3.0 |
| 3 | Menthol | 1.0 |
| 4 | White soft paraffin | 25.0 |
| 5 | Cetostearyl alcohol | 20.0 |
| 6 | Sodium laurlyl sulfate | 1.0 |
| 7 | Methyl papaben | 0.1 |
| 8 | Propyl paraben | 0.1 |
| 9 | Triethanolamine | 0.5 |
| 11 | Propylene glycol | 10.0 |
| 12 | Purified water | 31.3 |
| 13 | T. cacao dried and ground cells | 7.0 |

| | |
|---|---|
| Total | 100.0 |

### Example 12

### Efficacy of Cultured Cells

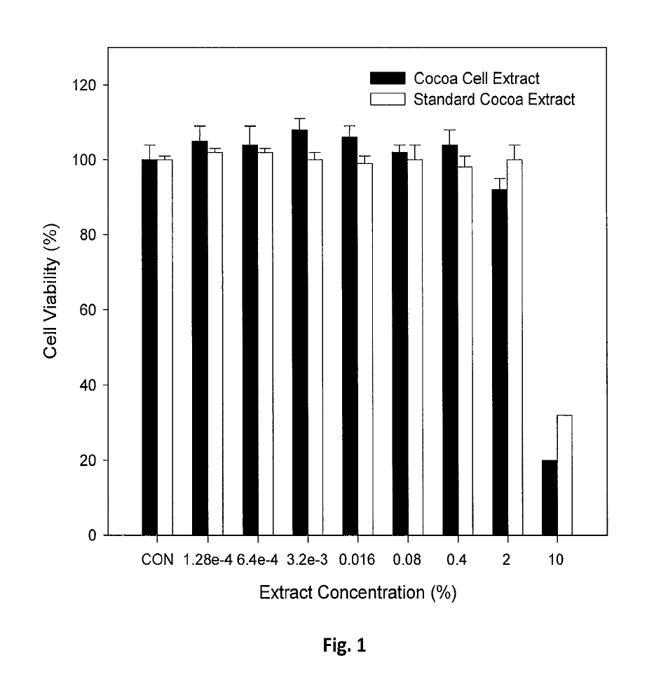

Example 12 describes a comparison of cell viability of cells exposed to a simulated immune response and treated with cultured cell extract (Cocoa Cell Extract) vs. a standard extract from cocoa beans (Standard Cocoa Extract). As seen in FIG. 1, extracts from cultured cells were more effective at lower concentrations (e.g., less than 2%) compared to the Standard Cocoa Extract. Thus, the cultured cells are more potent at lower concentrations and require less material to have the desired affect. This result is unexpected.

### Example 13

### Simulated IL-8

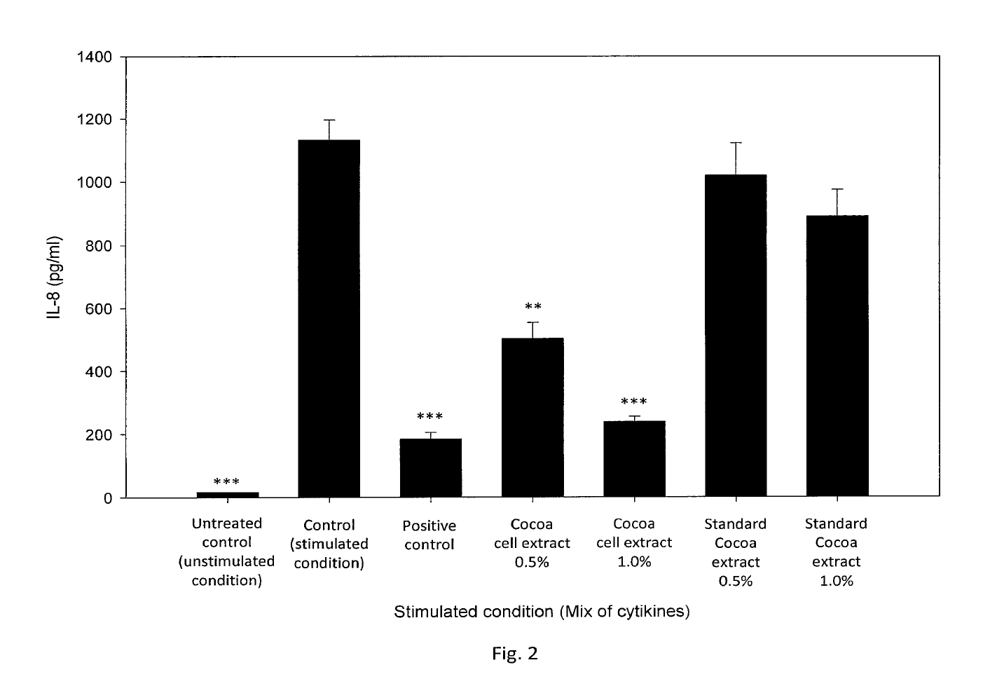

Example 13 illustrates a simulated condition of inflammation. The simulated inflammation includes a negative control (un-stimulated condition) and a first positive control (simulated condition) where inflammation is induced but no inhibition of the control. A second positive control inhibits the simulated inflammation response. The test samples include extract from cultured cells at 0.5% and 1.0% compared to a standard extract at the same concentrations. As shown in FIG. 2, the cultured cells had a substantially better ability to inhibit the simulated inflammation, thereby demonstrating the anti-inflammation properties of the cultured cells of the invention. The cultured cells approach the potency of the positive control at 1%. Even the 0.5% concentration is substantially better than both concentrations of standard extract. The asterisks in FIG. 2 correspond to the following statistical analysis: Student t-Test, *p<0.05, **p<0.01 and ***p<0.001.

Although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention

1. A dermatological composition for inhibiting or treating inflammation or aging of skin, the composition comprising:
a dermatologically compatible component; and
cultured callus cells or an extract thereof, the cultured callus cells being derived from a Theobroma cacao plant and including one or more anti-oxidant or anti-inflammatory compounds selected from the group consisting of, (−)-epicatechin, (+)-catechin, procyanidins, quercetin, isoquercetin (quercetin 3-O-glucoside), quercetin 3-O-arabinose, naringenin, and combinations of these,
wherein the extract or a lysate of the cultured callus cells inhibits expression of at least one inflammatory cytokine in an inflammation-induced cell sample.

2. (canceled)

3. The dermatological composition of claim 1, wherein the dermatological component is combined with the cultured callus cells or extract thereof to form a suspension, emulsion, paste, gel, cream, lotion, powder, soap, surfactant-containing cleanser, oil, powder foundation, emulsion foundation, wax foundation, or spray.

4. The dermatological composition of claim 1, wherein the composition includes the cultured callus cells in a concentration in a range from 0.1%-20% by weight.

5. The dermatological composition of claim 1, wherein the composition includes the extract of the cultured callus cells in a concentration in a range from 0.0001%-10% by weight.

6. The dermatological composition as in claim 1, wherein the cultured callus cells are derived from at least one of floral tissue selected from the group consisting of petals, sepals, staminodes, and combinations thereof or a non-floral vegetative tissue selected from the group consisting of nodes, internodes, young leaves, mature leaves, stems, roots, and combinations thereof.

7. (canceled)

8. The dermatological composition of claim 1, wherein the composition is formulated as a paste, cream, or gel, the composition including animal oil, plant oil, wax, paraffin, starch, tragacanth, cellulose derivatives, polyethylene glycol, silicone, bentonite, silica, talc, or zinc oxide.

9. A method of making a dermatological composition, comprising:
culturing isolated Theobroma cacao callus cells or obtaining an extract from the cultured Theobroma cacao callus cells; and
combining the cultured Theobroma cacao callus cells or the extract thereof with at least one dermatological component to yield the dermatological composition,
the isolated Theobroma cacao callus cells being selected to produce at least 5% polyphenols such that the extract or a lysate of the cultured Theobroma cacao callus cells inhibits expression of at least one inflammatory cytokine in an inflammation-induced cell sample.

10. The method of claim 9, wherein the dermatological composition is formulated as a solution, suspension, emulsion, paste, gel, cream, lotion, powder, soap, surfactant-containing cleanser, oil, powder foundation, emulsion foundation, wax foundation, or spray.

11. The method according to claim 9, wherein the cultured Theobroma cacao callus cells are extracted using an extraction solvent selected from the group consisting of water, absolute or aqueous lower alcohol containing 1-4 carbons, acetone, ethyl acetate, butyl acetate, dichloromethane (CH₂Cl₂), chloroform, hexane and 1,3-butylene glycol.

12. (canceled)

13. The method of claim 9, wherein the isolated Theobroma cacao callus cells are derived from at least one of floral tissue selected from the group consisting of petals, sepals, staminodes, and combinations thereof or a non-floral vegetative tissue selected from the group consisting of nodes, internodes, young leaves, mature leaves, stems, roots, and combinations thereof.

14. A method for treating skin, comprising:
identifying skin in need of treatment for inflammation or aging; and
topically applying to the skin a dermatological composition comprising a dermatologically compatible component and cultured Theobroma cacao callus cells or an extract thereof, the cultured callus cells or extracts including anti-oxidants or anti-inflammatory compounds,
the cultured Theobroma cacao callus cells being selected to produce at least 5% polyphenols and less than 1% anthocyanins.

15. The method as in claim 14, wherein the dermatological composition is formulated as a solution, suspension, emulsion, paste, gel, cream, lotion, powder, soap, surfactant-containing cleanser, oil, powder foundation, emulsion foundation, wax foundation, or spray.

16. The method as in claim 14, wherein the composition includes cultured cells in a concentration in a range from 0.1%-20% by weight.

17. The method of claim 14, wherein the composition includes an extract of Theobroma cacao callus cells from cell culture in a concentration in a range from 0.0001%-10% by weight.

18. The method of claim 14, wherein the composition is formulated as at least one of:
a paste, cream or gel, the composition including animal oil, plant oil, wax, paraffin, starch, tragacanth, cellulose derivatives, polyethylene glycol, silicone, bentonite, silica, talc, or zinc oxide; or
a powder or spray, the composition including lactose, talc, silica, aluminum hydroxide, calcium silicate or polyamide powder.

19. (canceled)

20. The method of claim 14, wherein the composition includes a solvent, solubilizer, or emulsifier, selected from the group of ethanol, isopropanol, ethyl carbonate, ethyl acetate, benzyl alcohol, benzyl benzoate, propylene glycol, 1,3-butylglycol oil, glycerol aliphatic ester, polyethylene glycol or fatty acid ester of sorbitan.

21. The dermatological composition of claim 1, wherein the at least one inflammatory cytokine is selected from the group consisting of MMP-2, MMP-9, IL-1β, IL-8, IL-18, IL-22, IL-23, IL-31, and MMP-1.

22. The dermatological composition of claim 1, wherein the extract inhibits expression of IL-8 in HDF cells treated with LPS by at least 50%, compared to treated control cells, at a concentration less than or equal to 1%.

23. The dermatological composition of claim 1, wherein the cultured callus cells or extract thereof comprises less than 1% anthocyanins.

24. The dermatological composition of claim 1, wherein at least one of:
the cultured callus cells comprise at least 5% polyphenols;
the cultured callus cells are selected to yield greater than 200 mg/L PCV of procyanidins; or
the cultured callus cells are selected to yield greater than 100 mg of procyanidins per liter of cell culture.
